(19) United States
(12) Patent Application Publication
(10) Pub. No.: US 2003/0211456 A1
(43) Pub. Date: Nov. 13, 2003

(54) Method of profiling a plant extract

(76) Inventors: Vassilios Papadopoulos, North Potomac, MD (US); Katy Drieu, Paris (FR)

Correspondence Address: Brian R Morrill, Biomeasure Incorporated, 27 Maple Street, Milford, MA, 01757, US

(21) Appl. No.: 10/240,052

(22) PCT Filed: Mar. 27, 2001
(86) PCT No.: PCT/US01/09832

(51) Int. Cl. C12Q 1/68; A61K 35/78; C12Q 1/00

(52) U.S. Cl. 435/4; 435/6; 424/752

(57) ABSTRACT

The present invention is directed to a method of establishing an “identity” of Ginkgo biloba leaves or isolated ginkgolide B (GKB) or another component of the extract of Ginkgo biloba leaves, by obtaining a “gene regulation profile”. The invention is also directed to a method of verifying the identity of a Ginkgo biloba extract by comparing the gene regulation profile of a Gingko biloba extract of unknown or questionable origin with the gene regulation pofile of a Gingko biloba extract of known origin. The present invention is further directed to a method of establishing a gene expression profile of Ginkgolide A, Ginkgolide B or any other component isolated from a Ginkgo biloba extract, more particularly, from EGB 761®.

### STATEMENT REGARDING FEDERALLY SPONSORED RESEARCH

The present invention was sponsored in part by grants ES-07747 NIEHS, from the National Institutes of Health and, thus, the U.S. government may have certain rights in the present invention.

### BACKGROUND OF THE INVENTION

The present invention is directed to a method of establishing an “identity” of Ginkgo biloba leaves or isolated ginkgolide B (GKB), a component of the extract of Ginkgo biloba leaves, by obtaining a “gene regulation profile”. The invention is also directed to a method of verifying the identity of a Ginkgo biloba extract by comparing the gene regulation profile of a Gingko biloba extract of unknown or questionable origin with the gene regulation profile of a Gingko biloba extract of known origin. The term “known origin” refers to the commercial source of the extract. A preferred aspect of the present invention is where the Ginkgo biloba extract of known origin is EGB 761®, produced and marketed by IPSEN of Paris, France. More particularly, this invention is directed to a method for determining the authenticity of an extract of unknown origin purporting to be EGB 761®. The present invention is further directed to a method of establishing a gene expression profile of Ginkgolide A, Ginkgolide B or any other component isolated from a Ginkgo biloba extract, more particularly, from EGB 761®.

Pharmaceutical manufacturing is based on control over the composition and the consistency of the biological activity profile of a manufactured batch. This standardization and control provides reproducible material in the predictable and consistent treatment of patients. Such use of standardization and control to guard against the marketing of counterfeit extracts purporting to be EGB 761® is beneficial to patients since it assures patients that they are obtaining/receiving an extract with a particular biological activity profile.

Ginkgo biloba is one of the most ancient trees, and extracts from its leaves have been used in traditional medicine for several hundred years. There are numerous studies describing the beneficial effects of Ginkgo biloba extracts on patients with disturbances in vigilance, memory, and cognitive functions associated with aging and senility, and on those with all types of dementias, mood changes, and the ability to cope with daily stressors. A standardized extract of Ginkgo biloba leaves, termed EGB 761®, has been used in most of these studies. This extract is also known to have cardioprotective effects (DeFeudis F. V. Ginkgo biloba extract (EGB 761®): from chemistry to clinic. Ullstein Medical, Wisbaden, Germany. 400 pp. 1998; Tosaki, A., Droy-Lefaix, M. T., Pali, T., and Das, D. K., Free Rad. Biol. Med., 14: 361-370, 1993). These effects have been attributed, at least in part, to the free radical scavenging properties of EGB 761®, probably due to the presence of flavonoid or terpenoid constituents in the extract. Recent in vivo and in vitro studies demonstrated that the terpene constituents of EGB 761®, ginkgolides and bilobalide, have anti-oxidant properties (Pietri, S., Maurelli, E., Drieu, K., and Culcasi, M., J. Mol. Cell. Cardiol., 29: 733-742, 1997; Yao, Z., Boujrad, N., Drieu, K., and Papadopoulos, V., Adv. Ginkgo Biloba Res. 7: 129-138, 1998). Other studies of EGB 761® have reported medicinal value of the product in the treatment of a variety of clinical disorders including cerebrovascular and peripheral vascular insufficiencies associated with aging and senility. See e.g., Ginkgo biloba Extract (EGB 761®) Pharmacological Activities and Clinical Applications, DeFeudis, F. V., Eds, Elsevier, 1991; and Ullstein Medical 1998, Ginkgo biloba extract (EGB 761®), Eds. Wiesbaden, DeFeudis, F. V. The extract contains 24% ginkgo-flavone glycosides, 6% terpene lactones (ginkgolides and bilobalide), about 7% proanthocyanidins and several other constituents. See Boralle, N., et al., In: Ginkgolides, Chemistry, Biology, Pharmacology and Clinical perspectives, Ed: Braquet, P., J. R. Prous Science Publishers, 1988.

More frequently, counterfeit formulations purporting to be EGB 761® are being placed in the stream of commerce. Such counterfeits do not possess the same composition of components that constitute authentic EGB 761®. Patients who obtain counterfeit EGB 761®, believing that the counterfeit is authentic, are being deprived the benefit of EGB 761®'s full range of biological activity. Further, the good will associated with EGB 761® is being eroded. Hence, there is a need for a method of establishing the biological activity profile of EGB 761®, which can then be used to compare with the biological activity profile of a counterfeit EGB 761 to screen such counterfeits from the marketplace.

### SUMMARY OF THE INVENTION

The present invention is directed to a method of establishing a gene regulation profile of a Gingko biloba extract or a component of the Ginkgo biloba extract, which comprises the steps of:

obtaining at least one batch of untreated cells;

treating a first batch of cells with an extract of Ginkgo biloba or a component of the Ginkgo biloba extract to obtain a treated batch of cells;

quantifying an affect on the expression of one or more genes of the treated cells to obtain a quantity of affected genes; and

comparing the quantity of affected genes with a quantity of genes of cells not treated with Ginkgo biloba or a component of the Ginkgo biloba extract to obtain the gene regulation profile of the Ginkgo biloba extract or a component of the Ginkgo biloba extract.

A preferred method of the foregoing method is where the quantifying step comprises:

isolating poly A+ RNA from the treated batch of cells to obtain treated poly A+ RNA;

isolating poly A+ RNA from a batch of untreated cells to obtain untreated poly A+ RNA;

generating labeled cDNA probes from the treated poly A+ RNA to obtain treated labeled cDNA probes;

generating labeled cDNA probes from the untreated poly A+ RNA to obtain untreated labeled cDNA probes;

hybridizing the treated cDNA probes to an array having one or more cDNA to obtain a treated hybridized array of cDNA;

hybridizing the untreated cDNA probes to an array having one or more cDNA to obtain an untreated hybridized array of cDNA;

quantifying each of the cDNA of the treated hybridized array of cDNA to obtain quantities of treated cDNA;

quantifying each of the cDNA of the untreated hybridized array of cDNA to obtain quantities of untreated cDNA; and

comparing the quantities of each of the treated cDNA with the quantities of untreated cDNA to obtain the gene regulation profile.

A preferred method of the immediately foregoing method is where the cells are MDA-231 cells; the Ginkgo biloba extract is EGB 7610®; and the array is a gene chip having multiple genes.

A preferred method of the immediately foregoing method is where the gene regulation profile of EGB 761® comprises increased expression of c-Myc protooncogene, and decreased expression of the following genes: prothymosin-α, CDK2, p55CDC, myeloblastin p120 proliferating-cell nuclear antigen, NET1, ERK2, Adenosine A2A Receptor, Flt3 ligand, Grb2, Clusterin, RXR-β, Glutathione S-transferase P, N-Myc, TRADD, SGP-2, NIP-1, Id-2, ATF-4, ETR101, ETR-103, macrophage colony-stimulating factor-1, heparin-binding EGF-like growth factor, hepatocyte growth factor-like protein, inhibin α, CD19 B-lymphocyte antigen, L1CAM, β-catenin, integrin subunit α3, integrin subunit α4, integrin subunit α6, integrin subunit β5, integrin subunit αM, APC, PE-1, RhoA, c-Jun, prothymosin-α, CDK2, p55CDC and myeloblastin.

A preferred method of the immediately foregoing method is where the gene regulation profile of EGB 761® is about

c-Myc=+75%,

c-Jun=−78%,

RhoA=−93%,

APC=−59%,

PE-1=−42%,

Prothymosin-α=−79%,

Myeloblastin=−66%,

p55CDC=−63%,

p120 Proliferating-cell Nuclear Antigen=−68%,

CDK2=−83%,

NET1=−55%,

ERK2=−46%,

Adenosine A2A Receptor=−40%,

Flt3 ligand=−58%,

Grb2=−70%,

Clusterin=−54%,

RXR-β=−55%,

Glutathione S-transferase P=−39%,

N-Myc=−74%,

TRADD=−51%,

NIP-1=−40%,

Id-2=−65%,

ATF4=−42%,

ETR103=−65%,

ETR101=−60%,

CD19 B-lymphocyte Antigen=−62%,

L1CAM=−72%,

β-catenin=−58%,

Integrin Subunit αM=−41%,

Integrin Subunit β5=−55%,

Integrin Subunit α4=−49%,

Integrin Subunit α3=−77%,

Integrin Subunit α6=−53%,

Macrophage Colony-stimulating Factor-1 (CSF-1)=−31%,

Heparin-binding EGF-like Growth Factor (HB-EGF)=−62%,

Hepatocyte Growth Factor-like Protein (HGFLP)=−81%, and

Inhibin α=−69%,

wherein the percentages shown can be ±20%.

A preferred method of any of the foregoing methods is where the cells are MDA-231 cells; the component of the Ginkgo biloba extract is Ginkgolide B; and the array is a gene chip having multiple genes.

In another aspect, the present invention provides a method of verifying the identity of a Ginkgo biloba extract which comprises the steps of:

obtaining a gene regulation profile of the Gingko biloba extract to obtain a gene regulation profile;

obtaining a gene regulation profile of EGB 761® to yield an EGB 761® gene regulation profile;

comparing the gene regulation profile of the Gingko biloba extract with the EGB 761® gene regulation profile;

determining whether the values of the gene regulation profile of the Ginkgo biloba extract is within ±10% of the values of the EGB 761® gene regulation profile to obtain verification of the identity of the Ginkgo biloba extract.

A preferred method of the immediately foregoing method is where the method of obtaining a gene regulation profile of the Ginkgo biloba extract and the EGB 761® gene regulation profile comprises the steps of:

isolating poly A+ RNA from the treated batch of cells to obtain treated poly A+ RNA;

isolating poly A+ RNA from a batch of untreated cells to obtain untreated poly A+ RNA;

generating labeled cDNA probes from the treated poly A+ RNA to obtain treated labeled cDNA probes;

generating labeled cDNA probes from the untreated poly A+ RNA to obtain untreated labeled cDNA probes;

hybridizing the treated cDNA probes to an array having one or more cDNA to obtain a treated hybridized array of cDNA;

hybridizing the untreated cDNA probes to an array having one or more cDNA to obtain an untreated hybridized array of cDNA;

quantifying each of the cDNA of the treated hybridized array of cDNA to obtain quantities of treated cDNA;

quantifying each of the cDNA of the untreated hybridized array of cDNA to obtain quantifies of untreated cDNA; and

comparing the quantities of each of the treated cDNA with the quantities of untreated cDNA to obtain the gene regulation profile.

### BRIEF DESCRIPTION OF THE DRAWINGS

FIG. 1. Transcriptional response to EGB 761® suggests an effect on genes involved in cell proliferation. Results shown represent quantitative analysis of the Atlas human cDNA expression array containing 588 PCR-amplified cDNA fragments (Clontech Inc.). mRNAs were obtained from control or EGB 761® (20 μg/ml) treated, for 48 h, MDA-231 cells. For normalizing the mRNA abundance, the densitometric values obtained from image analysis were normalized using the housekeeping genes provided in the array. Only consistent significant changes above 30% were considered.

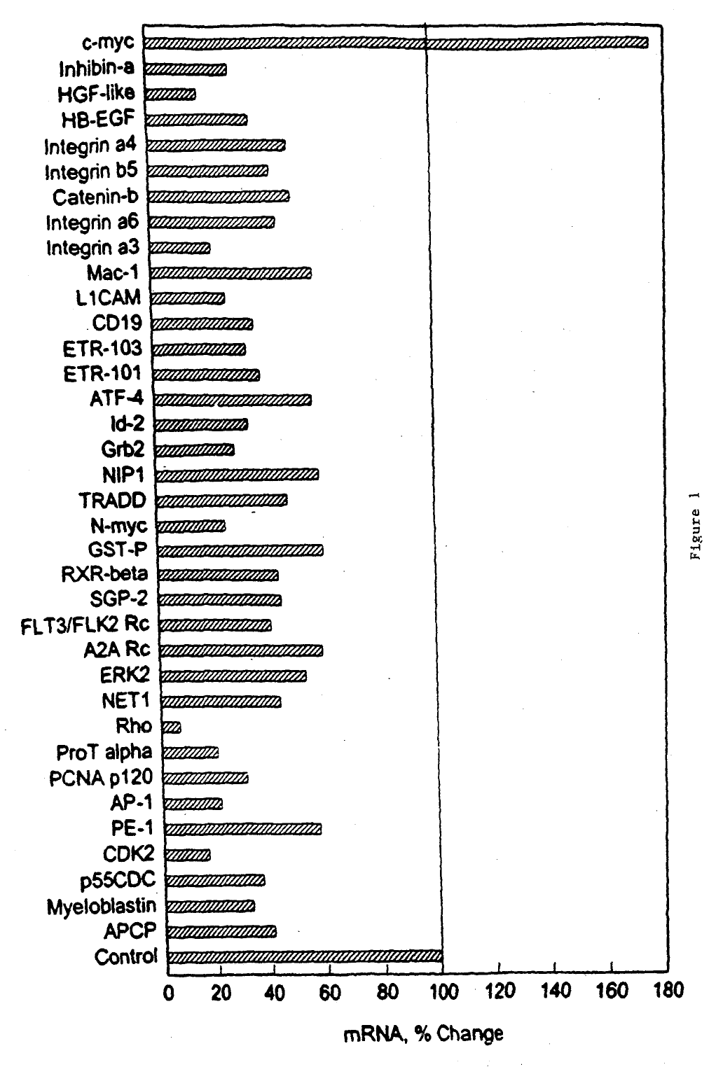

### DETAILED DESCRIPTION

The term “ginkgo terpenoid” includes all of the naturally occurring terpenes which are derived from the gymnosperms tree Ginkgo biloba as well as synthetically produced ginkgo terpenoids and pharmaceutically active derivatives and salts thereof and mixtures thereof. Examples of ginkgo terpenoids include ginkgolides. Examples of ginkgo terpenoids are disclosed in Ginkgolides, Chemistry, Biology, Pharmacology, and Clinical Perspectives, J. R. Provs. Science Publishers, Edited by P. Braguet (1988); F. V. DeFeudis, Ginkgo Biloba Extract (EGB 761®); Pharmacological Activities and Clinical Applications, Elsevier, Chapter II (1991).

The term “ginkgolide” as used herein include the various ginkgolides disclosed in the books cited above as well as non-toxic pharmaceutically active derivatives thereof. Examples of ginkgolide derivatives include tetrahydro derivatives, acetyl derivatives, and alkyl esters such as the monoacetate derivatives and triacetate derivatives disclosed in Okabe, et al., J. Chem. Soc. (c), pp. 2201-2206 (1967). Ginkgolide B has the following structure and as used herein, refers to isolated ginkgolide B:

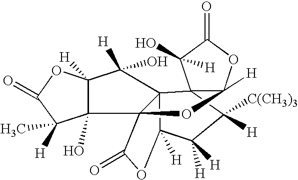

The term “Ginkgo biloba extract” as used herein includes a collection of natural molecules, including terpenoids, derived from the leaves of the Ginkgo biloba tree. Preferably, the extract is the specific formulation of Ginkgo biloba extract known as EGB 761®.

A gene expression profile of an extract of Ginkgo biloba or a component thereof can be obtained by methods known in the art. Traditionally such a profile was obtained by RNA Northern blot analysis or ribonuclease protection assay for each individual gene product. However, these assays were time consuming and took about 2-3 days to analyze each gene. Currently a gene expression profile can be established through the utilization of nucleic acid array technology such as the Atlas human cDNA expression array I from Clontech (Palo Alto, Calif.); GeneFilters Microarrays by Research Genetics (Huntsville, Ala.); and the Gene Expression Microarrays by Genome Systems, Inc. (St. Louis, Mo.). The Gene Filters Microarray are high density DNA arrays produced on 5 cm×7 cm membranes. At present there are four membranes available for human genes and one for rat genes. Each membrane contains approximately 5,000 sequences. Some of these sequences are known genes, while most sequences represent ESTs of unknown function. Research Genetics will soon make available gene arrays on the Affymetrix Gene chip platform, where the genes are immobilized on a silicon chip. In the case of silicon chips, the hybridization results (with the mRNA of choice) are detected by fluorescence and analyzed by pattern recognition compared to either fluorescence or radioactivity that can be used for the detection of the hybridization results in the membrane arrays.

Genome System's method utilizes the GEM technology where a collection of complementary DNA (cDNA) molecules that contain the genetic information from the biological systems of interest are deposited and bonded on a glass surface in an array format. Next, large portions from one half of the DNA's double strand are removed, thus activating the individual elements of the array, preparing them to react with their uniquely matched DNA counterparts in the cells being tested. GEM technology can fit 10,000 unique genes on a single array. GEM technology also uses a color coded technique to examine the difference in expression between two mRNA samples.

An array of cDNA will contain numerous animal, such as rat or human, preferably human, PCR-amplified cDNA fragments immobilized on a positively charged nylon membrane or a glass slide or a silicon chip or any other surface to be developed where a DNA/matrix interaction is allowed. A cell type of interest is treated with and without a Ginkgo biloba extract or a component thereof for about 48 hours. Poly A+ RNA is isolated from control and extract-treated cells. ³²P-labeled, fluorescent, chemiluminescent or colorimetric cDNA probes, preferably fluorescent or colorimetric labeling when using glass or silicon chip arrays, are generated from each poly A+ RNA and hybridized to the array according to the manufacturer's recommendations. Autoradiography is performed by exposing the blots to film at about −70° C. for 4-96 hr. Quantification of the hybridization is carried out using an imaging system, which can detect the fluorescence or chemiluminesence then capture the image and analyze the data, such as the SigmaGel software. Multiple exposures are used in order to detect genes expressed at low levels. The three internal controls, ubiquitin, G3PDH and β-actin are used to compare the relative expression levels of the detected gene products in the control and the extract-treated cells. Experimental variations are corrected using the ratios of gene expression versus the internal controls. The effect of the extract treatment on each gene product is expressed as % of control (untreated) cells.

An example of the foregoing type of gene expression profile is as follows. The Atlas human cDNA expression array I from Clontech (Palo Alto, Calif.) contains 588 human PCR-amplified cDNA fragments of 200-500 bp long immobilized on a positively charged nylon membrane. MDA-231 cells were treated with and without 20 μg/ml EGB 761® for 48 hours. Poly A+ RNA was isolated from control and EGB 761®-treated cells. ³²P-labeled cDNA probes were generated from each poly A+ RNA and hybridized to the Atlas array according to the manufacturer's recommendations. Autoradiography was performed by exposing the blots to X-OMAT AR film (Kodak, Rochester, N.Y.) at −70° C. for 4-96 hr. Quantification of the hybridization seen was carried out using the SigmaGel software (Jandel Scientific, San Rafael, Calif.). Multiple exposures were used in order to detect genes expressed at low levels. The three internal controls, ubiquitin, G3PDH and β-actin were used to compare the relative expression levels of the detected gene products in the control and EGB 761®-treated cells. Experimental variations were corrected using the ratios of gene expression versus the internal controls. The effect of the EGB 761® treatment on each gene product is expressed as % of control (untreated) cells. The results of this experiment, which is presented in Table I, show genes affected consistently, at a level above 30% of control, by the EGB 761® treatment. In summary, Table I shows that the treatment increased the expression of the c-Myc protooncogene and decreased the expression of 35 gene products, including oncogenes (AP-1, PE-1, RhoA, n-Myc), cell cycle regulators (CDK2, p55CDC, PCNA p120), signal transduction modulators (NET1, ERK2), apoptosis-related products (SGP-2, NIP1) receptors (A2A, RXR-beta, Grb2), transcription factors (Id-2, ATF-4, ETR101, ETR-103), growth factors (HB-EGF, HGF-like), and cell adhesion molecules (CD19, L1CAM, integrins α3, α4, α6, β5, Mac-1, β-catenin) which are directly involved in various pathways regulating cell proliferation.

Gene expression profiles can be established for Ginkgo biloba extracts of known origin and then can be compared with the gene expression profile of Ginkgo biloba extracts of unknown origin or extracts that purport to be a certain commercial extract. The comparison of the profiles can thus be used as a screening means to authenticate the origin of an extract.

TABLE I
Effect of EGB 761 ® on MDA-231 gene expression examined using the Atlas
human cDNA expression array as described under Nucleic Acid Arrays.

| | | | |
|---|---|---|---|
| Name | % Change | Function | Reference |

Oncogenes and Tumor Suppressers

| | | | |
|---|---|---|---|
| c-Myc | +75% | basic helix-loop-helix-leucine zipper transcription factor | (37) |
| | | Myc/Max heterodimers induce cell-cycle progression, apoptosis, and | |
| | | malignant transformation | |
| c-Jun | −78% | part of the AP-1 transcription factor that regulates genes involved in | (38) |
| | | cell proliferation | |
| RhoA | −93% | GTP-binding protein that is an important regulator of cell proliferation | (39) |
| | | RhoA inactivation inhibits HL60 cell proliferation | (40) |
| APC | −59% | APC mutations are associated with both hereditary and sporadic | (41) |
| | | colorectal cancers | (42) |
| | | a negative post-translational regulator of β-catenin | |
| PE-1 | −42% | transcription factor | (43) |

Cell Cycle Control Proteins

| | | | |
|---|---|---|---|
| Prothymosin-α | −79% | acidic nuclear protein that is upregulated in proliferation thymocytes, | (44) |
| | | lymphocytes from leukemia patients, and in malignant breast lesions | |
| Myeloblastin | −66% | a serine protease involved in leukemia cell differentiation | |
| p55CDC | −63% | similar to mitosis regulators CDC4 and CDC20 | (46) |
| | | expression positively correlated with cell proliferation status | |
| p120 Proliferating-cell | −68% | nucleolar protein expressed in proliferating cells | (47) |
| Nuclear Antigen | | a prognostic indicator for breast cancer patients and prostate | (48) |
| | | adenocarcinoma | |
| CDK2 | −83% | cyclin-dependent tyrosine kinase involved in progression through the | (49) |
| | | cell cycle | |
| | | cyclin E/Cdk2 inactivates the retinoblastoma tumor suppresser to | |
| | | allow the cell to progress to S phase | (50) |
| | | Vitamin D inhibition of LNCaP cell proliferation coincided with a | |
| | | reduction in Cdk2 activity | |

Intracellular Transducers

| | | | |
|---|---|---|---|
| NET1 | −55% | RhoA-specific guanine exchange factor | (51) |
| | | NIH3T3-transforming protein | |
| ERK2 | −46% | member of the extracellular signal-related protein kinase family | (52) |
| | | activated upon cell stimulation | |

Apoptosis-Related Proteins

| | | | |
|---|---|---|---|
| Adenosine A2A Receptor | −40% | G protein-coupled receptor involved in the cAMP signaling pathway | (53) |
| Fit3 ligand | −58% | ligand for the Fit3 cytokine receptor tyrosine kinase | (54) |
| | | induces proliferation of leukemic myeloid cells | |
| Grb2 | −70% | an adapter protein that links receptor tyrosine kinases to the | (55) |
| | | Ras/MAPK signaling pathway via its SH2 domain | |
| Clusterin | −54% | a glycoprotein associated with cell adhesion and apoptosis | (56, 57) |
| | | increased expression is linked to Alzheimer's disease | (58) |
| RXR-β | −55% | retiniod-activated transcription factor | (59) |
| | | inhibition of chondrocyte proliferation by retinoic acid causes a | (60) |
| | | reduction in RXR-β mRNA expression | |
| Glutathione S-transferase | −39% | a multi-drug resistance gene that is overexpressed in various human | (61, 62) |
| P | | tumors | (63) |
| | | chemical inhibition of GST-P inhibits proliferation of Jurkat T cells | |
| N-Myc | −74% | c-myc family member | (64) |
| | | associated with early-onset retinoblastoma | |
| TRADD | −51% | TNFR-associated death domain protein | (65) |
| | | involved in TNFR-induced cell growth and differentiation | |
| NIP-1 | −40% | originally described as a yeast nuclear transport protein | (66) |
| | | part of the translation initiation factor 3 (elF3) core complex | (67) |

DNA-Binding/Transcription Factors

| | | | |
|---|---|---|---|
| Id-2 | −65% | a member of the Id helix-loop-helix family of transcriptional inhibitors | (68) |
| | | involved in proliferation of human pancreatic cancer cells | |
| ATF4 | −42% | a member of the ATF/CREB family of transcription factors | (69) |
| | | regulates Ras-induced transformation of NIH3T3 cells | |
| ETR103 | −65% | a macrophage-associated immediate early gene | (70) |
| ETR101 | −60% | a lymphocyte-associated immediate early gene | (71) |

Cell Surface Antigens and Adhesion Molecules

| | | | |
|---|---|---|---|
| CD19 B-lymphocyte | −62% | B-lymphocyte integral membrane protein | (72) |
| Antigen | | expression is down-regulated during retinoid-inhibition of | |
| | | lymphoblastoid B-cell proliferation | |
| L1CAM | −72% | neural cell adhesion molecule | (73) |
| | | increased L1CAM expression is associated with high-grade migration | |
| | | of glioma cells | |
| β-catenin | −58% | involved in cadherin-mediated cell-cell interactions | (74) |
| | | interacts with the TCF/LEF transcription factors in the Wnt signaling | |
| | | pathway | |
| Integrin Subunits | | | |
| αM | −41% | mediates cellular adherence of human neutrophils with LFA-1β | (75) |
| | | α subunit of the elastase receptor | |
| β5 | −55% | β subunit of the vitronectin receptor (VR) | (76) |
| | | involved in cessation of oligodendrocyte proliferation | (77) |
| | | involved in murine retinal angiogenesis | (78) |
| α4 | −49% | cross-linking α4 integrins inhibits LB lymphoma cell proliferation | (79) |
| | | also involved in metastasis of melanoma and lymphoma cells | (80) |
| α3 | −77% | a functionally perturbing α3 integrin antibody inhibits human epithelial | (81) |
| | | cell proliferation | |
| α6 | −53% | overexpression of α6 integrin collaborates with ErbB2 to induce a | (82) |
| | | more malignant phenotype in NIH3T3 cells | |

Extracellular Signaling/Communication Proteins

| | | | |
|---|---|---|---|
| Macrophage Colony- | −31% | regulates the proliferation, differentiation, and survival of monocytes, | (83) |
| stimulating Factor-1 | | macrophages and their precursors | |
| (CSF-1) | | initiates a mitogenic signal that is required throughout G1 phase | |
| | | CSF-1 stably transfected ovarian granulosa cells exhibit enhanced | |
| | | cell proliferation | |
| Heparin-binding EGF-like | −62% | overexpressed in numerous human glioma cell lines and in a majority | (85) |
| Growth Factor (HB-EGF) | | of glioblastomas | |
| | | stimulates human glioma cell proliferation | |
| Hepatocyte Growth | −81% | a transmembrane protein tyrosine kinase found to be overexpressed | (86) |
| Factor-like Protein | | in hepatoblastoma and in human primary liver carcinoma | (87) |
| (HGFLP) | | induces proliferation and migration of murine keratinocytes | |
| Inhibin α | −69% | a member of the inhibin family of heterodimeric growth factors | (88) |
| | | inhibin α is a marker of trophoblastic neoplasia and is highly | (89) |
| | | expressed in virilizing adenomas | |

References:

37. Amati, B., Alevizopoulos, K., and Vlach, J. Myc and the Cell Cycle. Frontiers in Bioscience, 3: 250-268, 1998.

38. Huang, W. and Erikson, R. L. Signal Transduction. p. 161. Chapman and Hall, 1996.

39. Hu, W., Bellone, C. J., and Baldassare, J. J. RhoA stimulates p27(Kip) degradation through its regulation of cyclin E/CDK2 activity. J. Biol. Chem., 274: 3396-3401, 1999.

40. Aepfelbacher, M., Essler, M., Luber, D. Q., and Weber, P. C. ADP-ribosylation of the GTP-binding protein RhoA blocks cytoplasmic division in human myelomonocytic cells. Biochem. J., 308: 853-858, 1995.

41. Jeanteur, P. The role of APC in colon cancer: Zeroing in on Myc. Bulletin du Cancer (France), 85: 925928, 1998.

42. Munemitsu, S., Albert, I., Souza, B., Rubinfeld, B., and Polakis, P. Regulation of intracellular beta-catenin levels by the adenomatous polyposis coli (APC) tumor-suppressor protein. Proc. Natl. Acad. Sci. U.S.A., 92: 3046-3050, 1995.

43. Klemsz, M., Hromas, R., Raskind, W., Bruno, E., and Hoffman, R. PE-1, a novel ETS oncogene family member, localizes to chromosome 1q21-q23. Genomics, 20: 291-294, 1994.

44. Gomez-Marquez, J., Segade, F., Dosil, M., Pichel, J. G., Bustelo, X. R., and Freire, M. The expression of prothymosin alpha gene in T lymphocytes and leukemic lymphoid cells is tied to lymphocyte proliferation. J. Biol. Chem., 264: 8451-8454, 1989.

45. Bories, D., Raynal, M. C., Solomon, D. H., Darzynkiewicz, Z., and Cayre, Y. E. Down-regulation of a serine protease, myeloblastin, causes growth arrest and differentiation of promyelocytic leukemia cells. Cell, 59: 959968, 1989.

46. Kao, C. T., Lin, M., O'Shea-Greenfield, A., Weinstein, J., and Sakamoto, K. M. Over-expression of p55Cdc inhibits granulocyte differentiation and accelerates apoptosis in myeloid cells. Oncogene, 13: 1221-1229, 1996.

47. Landberg, G. and Roos, G. The Cell Cycle in Breast Cancer. APMIS, 105: 575-589, 1997.

48. Perlaky, L., Valdez, B. C., Busch, R. K., Larson, R. G., Jhiang, S. M., Zhang, W. W., Brattain, M., and Busch, H. Increased growth of NIH/3T3 cells by transfection with human p120 complementary DNA and inhibition by a p120 antisense construct. Cancer Res., 52: 428436, 1992.

49. Zhuang, S. H. and Burnstein, K. L. Antiproliferative Effect of 1alpha,25-dihydroxyvitamin D3 in Human Prostate Cancer Cell Line LNCaP Involves Reduction of Cyclin-dependent Kinase 2 Activity and Persistent G1 Accumulation. Endocrinology, 139:1197-1207, 1998.

50. Chan, A. M., Takai, S., Yamada, K., and Miki, T. Isolation of a novel oncogene, NET1, from neuroepithelioma cells by expression cDNA cloning. Oncogene, 12: 1259-1266, 1996.

51. Ahn, N. G. and Tolwinski, N. S. U0126: An Inhibitor of MKK/ERK Signal Transduction in Mammalian Cells. Promega Notes, 71: 4-13, 1999.

52. Coppee, F., Gerard, A. C., Denef, J. F., Ledent, C., Vassart, G., Dumont, J. E., and Parmentier, M. Early occurrence of metastatic differentiated thyroid carcinomas in transgenic mice expressing the A2a adenosine receptor gene and the human papillomavirus type 16 E7 oncogene. Oncogene, 13: 1471-1482, 1996.

53. Dehmel, U., Zaborski, M., Meierhoff, G., Rosnet, O., Bimbaum, D., Ludwig, W. D., Quentmeier, H., and Drexler, H. G. Effects of FLT3 ligand on human leukemia cells. I. Proliferative response of myeloid leukemia cells. Leukemia, 10: 261-270, 1996.

54. Benito, M., Valverde, A. M., and Lorenzo, M. IGF-I: a mitogen also involved in differentiation processes in mammalian cells. lnt.J Biochem. Cell Biol., 28: 499-510, 1996.

55. Chevalier, R. L. Effects of ureteral obstruction on renal growth. Semin. Nephrol., 15: 353-360, 1995.

56. Truong, L. D., Sheikh-Hamad, D., Chakraborty, S., and Suki, W. N. Cell Apoptosis and Proliferation in Obstructive Uropathy. Semin. Nephrol., 18: 641-651, 1998.

57. Choi-Miura, N. H. and Oda, T. Relationship between multifunctional protein “clusterin” and Alzheimer disease. Neurobiol. Aging, 17: 717-722, 1996.

58. Lotan, R. Retinoids and chemoprevention of aerodigestive tract cancers. Cancer Metastasis Rev., 16: 349-356, 1997.

59. Hagiwara, H., Inoue, A., Nakajo, S., Nakaya, K., Kojima, S., and Hirose, S. Inhibition of proliferation of chondrocytes by specific receptors in response to retinoids. Biochem. Biophys. Res. Commun., 222: 220-224, 1996.

60. Campos, L., Oriol, P., Sabido, O., and Guyotat, D. Simultaneous expression of P-glycoprotein and BCL-2 in acute myeloid leukemia blast cells. Leuk. Lymphoma., 27: 119-125, 1997.

61. Lacave, R., Coulet, F., Ricci, S., Touboul, E., Flahault, A., Rateau, J. G., Cesari, D., Lefranc, J. P., and Bernaudin, J. F. Comparative evaluation by semiquantitative reverse transcriptase polymerase chain reaction of MDR1, MRP and GSTp gene expression in breast carcinomas. Br. J. Cancer, 77: 694-702, 1998.

62. McCaughan, F. M., Brown, A. L., and Harrison, D. J. The effect of inhibition of glutathione S-transferase P on the growth of the Jurkat human T cell line. J. Pathol., 172: 357-362, 1994.

63. Kim, C. J., Chi, J. G., Choi, H. S., Shin, H. Y., Ahn, H. S., Yoo, Y. S., and Chang, K. Y. Proliferation not Apoptosis as a Prognostic Indicator in Retinoblastoma. Virchows Arch., 434: 301-305, 1999.

64. Newton, K., Harris, A. W., Bath, M. L., Smith, K. C., and Strasser, A. A dominant interfering mutant of FADD/MORT1 enhances deletion of autoreactive thymocytes and inhibits proliferation of mature T lymphocytes. EMBO J., 17: 706-718, 1998.

65. Gu, Z., Moerschell, R. P., Sherman, F., and Goldfarb, D. S. NIP1, a gene required for nuclear transport in yeast. Proc. Natl. Acad. Sci. U.S.A., 89: 10355-10359, 1992.

66. Phan, L., Zhang, X., Asano, K., Anderson, J., Vomlocher, H. P., Greenberg, J. R., Qin, J., and Hinnebusch, A. G. Identification of a translation initiation factor 3 (elF3) core complex, conserved in yeast and mammals, that interacts with elF5. Mol. Cell Biol., 18: 4935-4946, 1998.

67. Kleeff, J., Ishiwata, T., Friess, H., Buchler, M. W., Israel, M. A., and Korc, M. The helix-loop-helix protein ld2 is overexpressed in human pancreatic cancer. Cancer Res., 58: 3769-3772, 1998.

68. Mielnicki, L. M., Hughes, R. G., Chevray, P. M., and Pruitt, S. C. Mutated Atf4 suppresses c-Ha-ras oncogene transcript levels and cellular transformation in NIH3T3 fibroblasts. Biochem. Biophys. Res.Commun., 228: 586-595, 1996.

69. Shimizu, N., Ohta, M., Fujiwara, C., Sagara, J., Mochizuki, N., Oda, T., and Utiyama, H. A gene coding for a zinc finger protein is induced during 12-O-tetradecanoylphorbol-13-acetate-stimulated HL-60 cell differentiation. J. Biochem. (Tokyo.), 111: 272-277, 1992.

70. Shimizu, N., Ohta, M., Fujiwara, C., Sagara, J., Mochizuki, N., Oda, T., and Utiyama, H. Expression of a novel immediate early gene during 12-O-tetradecanoylphorbol-13-acetate-induced macrophagic differentiation of HL-60 cells. J. Biol. Chem., 266: 12157-12161, 1991.

71. Dolcetti, R., Zancai, P., Cariati, R., and Boiocchi, M. In vitro effects of retinoids on the proliferation and differentiation features of Epstein-Barr virus-immortalized B lymphocytes. Leuk.Lymphoma, 29: 269-281, 1998.

72. Tsuzuki, T., Izumoto, S., Ohnishi, T., Hiraga, S., Arita, N., and Hayakawa, T. Neural cell adhesion molecule L1 in gliomas: correlation with TGF-beta and p53. J. Clin. Pathol., 51: 13-17, 1998.

73. Young, C. S., Kitamura, M., Hardy, S., and Kitajewski, J. Wnt-1 induces growth, cytosolic beta-catenin, and Tcf/Lef transcriptional activation in Rat-1 fibroblasts. Mol Cell Biol., 18: 2474-2485, 1998.

74. Cai, T. Q. and Wright, S. D. Human leukocyte elastase is an endogenous ligand for the integrin CR3 (CD11b/CD18, Mac-1, alpha M beta 2) and modulates polymorphonuclear leukocyte adhesion. J. Exp. Med, 184: 1213-1223, 1996.

75. De Deyne, P. G., O'Neill, A., Resneck, W. G., Dmytrenko, G. M., Pumplin, D. W., and Bloch, R. J. The Vitronectin Receptor Associates with Clathrin-coated Membrane Domains Via the Cytoplasmic Domain of its beta5 Subunit. J. Cell Sci., 111: 2729-2740, 1998.

76. Milner, R. and Ffrench-Constant, C. A developmental analysis of oligodendroglial integrins in primary cells: changes in alpha v-associated beta subunits during differentiation. Development, 120: 3497-3506, 1994.

77. Friedlander, M., Theesfeld, C. L., Sugita, M., Fruttiger, M., Thomas, M. A., Chang, S., and Cheresh, D. A. Involvement of integrins alpha v beta 3 and alpha v beta 5 in ocular neovascular diseases. Proc. Nati. Acad. Sci. U.S.A., 93: 9764-9769, 1996.

78. Bittner, M., Gosslar, U., Luz, A., and Holzmann, B. Sequence Motifs in the Integrin alpha4 Cytoplasmic Tail Required for Regulation of In Vivo Expansion of Murine Lymphoma Cells. J. Immunol., 161: 5978-5986, 1998.

79. Holzmann, B., Gosslar, U., and Bittner, M. alpha 4 integrins and tumor metastasis. Curr.Top. Microbiol. Immunol., 231: 125-141, 1998.

80. Gonzales, M., Haan, K., Baker, S. E., Fitchmun, M., Todorov, I., Weitzman, S., and Jones, J. C. A Cell Signal Pathway Involving Laminin-5, alpha3beta1 Integrin, and Mitogen-activated Protein Kinase can Regulate Epithelial Cell Proliferation. Mol. Biol. Cell, 10: 259-270, 1999.

81. Falcioni, R., Antonini, A., Nistico, P., Di, S. S., Crescenzi, M., Natali, P. G., and Sacchi, A. Alpha 6 beta 4 and alpha 6 beta 1 integrins associate with ErbB-2 in human carcinoma cell lines. Exp. Cell Res., 236: 76-85, 1997.

82. Roussel, M. F. Regulation of cell cycle entry and G1 progression by CSF-1. Mol. Reprod. Dev., 46:11-18, 1997.

83. Keshava, N., Gubba, S., and Tekmal, R. R. Overexpression of Macrophage Colony-stimulating Factor (CSF-1) and its Receptor, c-fms, in Normal Ovarian Granulosa Cells Leads to Cell Proliferation and Tumorigenesis. J. Soc. Gynecol. Investig., 6: 41-49, 1999.

84. Mishima, K., Higashiyama, S., Asai, A., Yamaoka, K., Nagashima, Y., Taniguchi, N., Kitanaka, C., Kirino, T., and Kuchino, Y. Heparin-binding Epidermal Growth Factor-like Growth Factor Stimulates Mitogenic Signaling and is Highly Expressed in Human Malignant Gliomas. Acta Neuropathologica, 96: 322-328, 1998.

85. Zhu, M. and Paddock, G. V. Expression of the Hepatocyte Growth Factor-like Protein Gene in Human Hepatocellular Carcinoma and lnterleukin-6-induced Increased Expression in Hepatoma Cells. Biochim. Biophys. Acta, 1449: 63-72, 1999.

86. Wang, M. H., Dlugosz, A. A., Sun, Y., Suda, T., Skeel, A., and Leonard, E. J. Macrophage-stimulating protein induces proliferation and migration of murine keratinocytes. Exp. Cell Res., 226: 39-46, 1996.

87. Pelkey, T. J., Frierson, H. F., Mills, S. E., and Stoler, M. H. Detection of the alpha-subunit of Inhibin in Trophoblastic Neoplasia. Hum. Pathol., 30: 26-31, 1999.

88. Arola, J., Liu, J., Heikkila, P., Voutilainen, R., and Kahri, A. Expression of inhibin alpha in the human adrenal gland and adrenocortical tumors. Endocrine Res., 24: 865-867, 1998.

89. Kallakury, B. S., Sheehan, C. E., Rhee, S. J., Fisher, H. G., Kaufman, R. P. J., Rifkin, M. D., and Ross, J. S. The prognostic significance of proliferation-associated nucleolar protein p120 expression in prostate adenocarcinoma: A comparison with cyclins A and B1, Ki-67, proliferating cell nuclear antigen, and p34(cdc2). Cancer, 85: 1569-1576, 1999.

What is claimed is:

1. A method of establishing a gene regulation profile of a Gingko biloba extract or a component of the Ginkgo biloba extract, which comprises the steps of:
obtaining at least one batch of untreated cells;
treating a first batch of cells with an extract of Ginkgo biloba or a component of the Ginkgo biloba extract to obtain a treated batch of cells;
quantifying an affect on the expression of one or more genes of the treated cells to obtain a quantity of affected genes; and
comparing the quantity of affected genes with a quantity of genes of cells not treated with Ginkgo biloba or a component of the Ginkgo biloba extract to obtain the gene regulation profile of the Ginkgo biloba extract or a component of the Ginkgo biloba extract.

2. A method according to claim 1 wherein the quantifying comprises:
isolating poly A+ RNA from the treated batch of cells to obtain treated poly A+ RNA;
isolating poly A+ RNA from a batch of untreated cells to obtain untreated poly A+ RNA;
generating labeled cDNA probes from the treated poly A+ RNA to obtain treated labeled cDNA probes;
generating labeled cDNA probes from the untreated poly A+ RNA to obtain untreated labeled cDNA probes;
hybridizing the treated cDNA probes to an array having one or more cDNA to obtain a treated hybridized array of cDNA;
hybridizing the untreated cDNA probes to an array having one or more cDNA to obtain an untreated hybridized array of cDNA;
quantifying each of the cDNA of the treated hybridized array of cDNA to obtain quantities of treated cDNA;
quantifying each of the cDNA of the untreated hybridized array of cDNA to obtain quantities of untreated cDNA; and
comparing the quantities of each of the treated cDNA with the quantities of untreated cDNA to obtain the gene regulation profile.

3. A method according to claim 2, wherein the cells are MDA-231 cells; the Ginkgo biloba extract is EGB 761®; and the array is a gene chip having multiple genes.

4. A method according to claim 3 wherein the gene regulation profile of EGB 761® comprises increased expression of c-Myc protooncogene, and decreased expression of the following genes: prothymosin-α, CDK2, p55CDC, myeloblastin p120 proliferating-cell nuclear antigen, NET1, ERK2, Adenosine A2A Receptor, Flt3 ligand, Grb2, Clusterin, RXR-β, Glutathione S-transferase P, N-Myc, TRADD, SGP-2, NIP-1, Id-2, ATF-4, ETR101, ETR-103, macrophage colony-stimulating factor-1, heparin-binding EGF-like growth factor, hepatocyte growth factor-like protein, inhibin α, CD19 B-lymphocyte antigen, L1CAM, P-catenin, integrin subunit α3, integrin subunit α4, integrin subunit α6, integrin subunit β5, integrin subunit αM, APC, PE-1, RhoA, c-Jun, prothymosin-α, CDK2, p55CDC and myeloblastin.

5. A method according to claim 4 wherein the gene regulation profile of EGB 761® is about
c-Myc=+75%,
c-Jun=−78%,
RhoA=−93%,
APC=−59%,
PE-1=−42%,
Prothymosin-α=−79%,
Myeloblastin=−66%,
p55CDC=−63%,
p120 Proliferating-cell Nuclear Antigen=−68%,
CDK2=−83%,
NET1=−55%,
ERK2=−46%,
Adenosine A2A Receptor=−40%,
Flt3 ligand=−58%,
Grb2=−70%,
Clusterin=−54%,
RXR-β=−55%,
Glutathione S-transferase P=−39%,
N-Myc=−74%,
TRADD=−51%,
NIP-1=−40%,
Id-2=−65%,
ATF4=−42%,
ETR103=−65%,
ETR101=−60%,
CD19 B-lymphocyte Antigen=−62%,
L1CAM=−72%,
β-catenin=−58%,
Integrin Subunit αM=−41%,
Integrin Subunit β5=−55%,
Integrin Subunit α4=−49%,
Integrin Subunit α3=−77%,
Integrin Subunit α6=−53%,
Macrophage Colony-stimulating Factor-1 (CSF-1)=−31%,
Heparin-binding EGF-like Growth Factor (HB-EGF)=−62%,
Hepatocyte Growth Factor-like Protein (HGFLP)=−81%, and
Inhibin α=−69%,
wherein the percentages shown can be ±20%.

5. A method according to claim 2, wherein the cells are MDA-231 cells; the component of the Ginkgo biloba extract is Ginkgolide B; and the array is a gene chip having multiple genes.

6. A method of verifying the identity of a Ginkgo biloba extract which comprises the steps of:
obtaining a gene regulation profile of the Gingko biloba extract to obtain a gene regulation profile;
obtaining a gene regulation profile of EGB 761® to yield an EGB 761® gene regulation profile;
comparing the gene regulation profile of the Gingko biloba extract with the EGB 761® gene regulation profile;
determining whether the values of the gene regulation profile of the Ginkgo biloba extract is within ±10% of the values of the EGB 761® gene regulation profile to obtain verification of the identity of the Ginkgo biloba extract.

7. A method according to claim 6, wherein the method of obtaining a gene regulation profile of the Ginkgo biloba extract and the EGB 761® gene regulation profile comprises the steps of:
isolating poly A+ RNA from the treated batch of cells to obtain treated poly A+ RNA;
isolating poly A+ RNA from a batch of untreated cells to obtain untreated poly A+ RNA;
generating labeled cDNA probes from the treated poly A+ RNA to obtain treated labeled cDNA probes;
generating labeled cDNA probes from the untreated poly A+ RNA to obtain untreated labeled cDNA probes;
hybridizing the treated cDNA probes to an array having one or more cDNA to obtain a treated hybridized array of cDNA;
hybridizing the untreated cDNA probes to an array having one or more cDNA to obtain an untreated hybridized array of cDNA;
quantifying each of the cDNA of the treated hybridized array of cDNA to obtain quantities of treated cDNA;
quantifying each of the cDNA of the untreated hybridized array of cDNA to obtain quantities of untreated cDNA; and
comparing the quantities of each of the treated cDNA with the quantities of untreated cDNA to obtain the gene regulation profile.
